SEQUENCE LISTING (9 sequences)

SEQ ID NO: 1 - DNA, 21 bp, artificial
  PCR primer-PR2
tcattgggaggccagagcatc

SEQ ID NO: 2 - DNA, 30 bp, artificial
  PCR primer-PR3
atatggtacagcaacaaagcatttgacata

SEQ ID NO: 3 - DNA, 25 bp, artificial
  PCR primer-PR7
gaattgtttgctgtggatgcatgta

SEQ ID NO: 4 - DNA, 20 bp, artificial
  PCR primer-K14CreR
ttcctcaggagtgtcttcgc

SEQ ID NO: 5 - DNA, 20 bp, artificial
  PCR primer-K14CreF
gtccatgtccttcctgaagc

SEQ ID NO: 6 - DNA, 19 bp, artificial
  Top2Beta-shRNA
gcccccgttatatcttcac

SEQ ID NO: 7 - DNA, 53 bp, artificial
  Top2Beta-shRNA Duplex DNA
gcccccgttatatcttcacttcaagagagtgaagatataacgggggctttttc

SEQ ID NO: 8 - DNA, 19 bp, artificial
  Control-shRNA
gcgcgcgttaaatcttcac

SEQ ID NO: 9 - DNA, 54 bp, artificial
  Control-shRNA Duplex DNA
tgcgcgcgttaaatcttcacttcaagagagtgaagatttaacgcgcgctttttc

(19) United States
(12) Patent Application Publication
(10) Pub. No.: US 2011/0275596 A1
(43) Pub. Date: Nov. 10, 2011

(54) METHODS FOR TREATING NEOPLASIA AND FOR IDENTIFYING COMPOSITIONS USEFUL IN SUCH THERAPY

(75) Inventors: Leroy F. Liu, Bridgewater, NJ (US); Yi Lisa Lyu, Warren, NJ (US); Anna M. Azarova, Winchester, MA (US); Johnson Yiu-Nam Lau, Newport Beach, CA (US)

(73) Assignee: UNIVERSITY OF MEDICINE AND DENTISTRY OF NEW JERSEY, Somerset, NJ (US)

(21) Appl. No.: 12/967,261

(22) Filed: Dec. 14, 2010

Related U.S. Application Data
(63) Continuation of application No. 12/664,811, now abandoned.
(60) Provisional application No. 60/944,333, filed on Jun. 15, 2007.

(51) Int. Cl. A61K 31/69; G01N 33/573; A61P 35/02; A61K 31/496; A61K 31/437; A61P 35/00

(52) U.S. Cl. 514/64; 514/292; 514/252.11; 435/7.6

(57) ABSTRACT

Various methods for treating a patient with neoplasia are disclosed, in particular, methods using topoisomerase Ila-preferential poisons, methods using a combination of a topoisomerase Illi-preferential inhibitor and a topoisomerase II poison, and methods using a combination of a topoisomerase II poison and a proteasome inhibitor are disclosed. Novel topoisomerase Ila-preferential poisons are disclosed, particularly, several novel 13-carboline derivatives are identified. Methods for identifying the novel topoisomerase Ila-preferential poisons and methods for identifying the novel topoisomerase EP-preferential inhibitors are also provided herein.

### FIELD OF THE INVENTION

This invention relates to therapeutic methods for treating a patient with neoplasia by administering to the patient a therapeutically effective amount of a topoisomerase IIα preferential poison, and methods to identify such a compound. This invention also provides therapeutic methods for treating neoplasia, including an administration of a therapeutically effective amount of a topoisomerase II inhibitor that can reduce topoisomerase IIβ-mediated damages followed by the administration of a therapeutically effective amount of a non-selective topoisomerase II poison to the patient, and methods of identifying a compound for use as the inhibitor. Also this invention provides therapeutic methods for treating neoplasia, including co-administration of a therapeutically effective amount of a proteasome inhibitor with a therapeutically effective amount of a topoisomerase II poison, that can reduce topoisomerase IIβ-mediated damage.

### BACKGROUND OF THE INVENTION

Topoisomerase II-targeting anticancer drugs such as etoposide, doxorubicin and mitoxantrone are among the most widely used chemotherapeutic agents in the treatment of various human cancers and leukemia. However, major side effects can limit their effective use. For example, treatment-related acute myeloid leukemia (t-AML) is well known to be associated with etoposide-based chemotherapy. Life-threatening cardiotoxicity is another well known toxicity associated with doxorubicin-based therapy. At the present time, there is no effective strategy to deal with these major toxic side effects of topoisomerase II-targeting drugs.

Two topoisomerase II (“Top 2”) isozymes, topoisomerase IIα (“Top2α”) and topoisomerase IIβ (“Top2β”), have been identified in mammalian cells. The Top2α isozyme is a homodimer with a monomer molecular weight of 170 kDa, while Top2β isozyme, encoded by the gene on chromosome 3p24, is a homodimer with a molecular weight of 180 kDa. The enzymatic activity of Top2β is the same as Top2α. In fact, the two isozymes show about 70% sequence identity.

Currently, all clinically used Top2-targeting drugs are known to target both Top2 isozymes, Top2α and Top2β, more or less indiscriminately and non-selectively. All these drugs act by the same mechanism. They poison both Top2 isozymes indiscriminately through stabilizing their respective covalent reaction intermediates, the cleavable/cleavage complexes. The word “poison” is often used herein to indicate this specific mechanism of Top2 inhibition. This inhibition mechanism is summarized in FIG. 1B, in which a G-segment (gate segment) is bound by Top2. In the absence of ATP, Top2 exists in the open clamp conformation (demonstrated by the pair of jaws at the top of the Top2 homodimer). Upon ATP binding, Top2 is stabilized in the closed-clamp conformation and performs the cleavage and strand-passing reaction. Upon ATP hydrolysis, Top2 returns to its original conformation and another cycle of strand-passing can resume. To date, all clinically used Top2-targeting drugs such as etoposide and doxorubicin block the re-ligation reaction, resulting in accumulation of the cleavage complex (in ATP-bound closed-clamp conformation).

Top2α has been known to be a cell proliferation marker, being highly expressed in proliferating cells in late S/G2 phase of the cell cycle and absent in quiescent or differentiated cells. Top2α has also been identified to be the chromosome scaffold protein which together with condensin to form the chromosome axis. Further, Top2α has been suggested to be important for cell cycle events such as DNA replication, chromosome condensation and sister-chromatid separation.

In many instances, tumor cells are known to express even higher levels of Top2α. For example, Top2α is often expressed at very high levels in breast cancer cells that are Her2/neu-positive due to co-localization of the Top2α gene and the Her2/Neu gene on the same chromosome 17q21 locus. At present, the reasons for the highly elevated expression of Top2α in tumor cells are not completely understood. It is speculated that Top2α is rapidly degraded in normal proliferating cells upon exiting mitosis. As contrast, many tumor cells are defective in Top2α degradation, resulting in elevated Top2α expression throughout the cell cycle.

In addition, it is known that the Top2α gene is negatively regulated by the tumor suppressor p53, which occur in many tumors. It is found that mutations of p53 can lead to elevated expression of Top2α, which suggests that Top2α is not only a cell proliferation marker but also a tumor marker.

By contrast, Top2β has been suggested to participate in gene transcription and is expressed at similar levels in proliferating and quiescent cells.

At present, all clinically relevant Top2 poisons (e.g. doxorubicin, etoposide, epirubicin and mitoxantrone) poison both Top2α and Top2β indiscriminately. Applicants discoverted that Top2α-poisoning is mainly associated with the antitumor activity of these poisons, while Top2β-poisoning is associated with the major tissue toxicities of currently used Top2-targeting drugs. For example, Applicants found that etoposide-induced carcinogenesis is Top2β-dependent, suggesting a major role of Top2β poisoning in etoposide-induced secondary leukemia (i.e. t-AML). In addition, Applicants discovered that doxorubicin-induced DNA damage is Top2β-mediated in cardiomyocytes, suggesting a major role of Top2β poisoning in doxorubicin cardiotoxicity. The Applicants discovered that Top2α-poisoning is primarily responsible for the tumor cell killing activity of etoposide, while Top2β-targeting by etoposide does not contribute significantly to the tumor cell killing activity of etoposide. Applicants' new discoveries highlight that it is highly desirable to develop Top2α isozyme-preferential poisons. These Top2α preferential agents have high anti-neoplastic activity with minimal side effects such as secondary leukemia and tissue toxicities (e.g. cardiotoxicity and skin lesions).

### SUMMARY OF THE INVENTION

Applicants discovered that human Top2α isozyme represents a distinct molecular target for development of anticancer drugs. Compounds that preferentially poison human Top2α isozyme compared to human Top2β isozyme should exhibit reduced side effects such as second malignancies and tissue toxicities associated with non-preferential Top2 poisons.

Applicants discovered unexpectedly that there exists a class of Top2 poisons that preferentially poison the Top2α isozyme compared to the Top2β isozyme. Applicants find that the toxic side effects associated with current Top2-based therapies can be reduced or even eliminated by administering these Top2α preferential poisons to patients with neoplasia.

In one aspect, this invention is directed to a method for treating a patient with neoplasia comprising administering to the patient a therapeutically effective amount of a Top2 poison that preferentially poisons the Top2α isozyme as compared to the Top2β isozyme.

Another aspect of this invention is a method for treating a patient with a combination of a therapeutically effective amount of a Top2 inhibitor and a therapeutically effective amount of a Top2 poison. This method comprises (a) administering a therapeutically effective amount of a Top2 inhibitor to a patient with neoplasia, wherein the inhibitor causes preferential degradation of the Top2β isozyme over Top2α isozyme; (b) administering a therapeutically effective amount of at least one Top2 poison to the patient; wherein the Top2 inhibitor is administered at least 2 hours prior to administration of the Top2 poison.

Another aspect of this invention is a method for treating a patient with neoplasia comprising: administering a therapeutically effective amount of a Top2 poison and a therapeutically effective amount of a proteasome inhibitor to said patient.

Another aspect of this invention is a method for identifying a compound for use as a Top2α preferential poison. The method comprises: (a) evaluating the compound for its ability to poison Top2α isozyme; (b) evaluating the compound for its ability to poison Top2β isozyme; and (c) selecting a compound that preferentially poisons the Top2α isozyme over the Top2β isozyme.

The present invention also provides methods for identifying Top2β inhibitors through high-throughput screening, wherein said Top2β inhibitors can interfere with Top2β-mediated tissue damage to avoid toxic side effects of Top2-based chemotherapy.

### BRIEF DESCRIPTION OF THE DRAWINGS

FIG. 1. Inhibition mechanisms of Top2-targeting drugs on Top2 isozymes: trapping of the Top2 cleavage complex in the catalytic cycle.

FIG. 2. Results of Applicants' studies employing neutral comet assay to assess the roles of Top2 isozymes in the generation of etoposide (VP-16)-induced DNA double-strand breaks in MEFs.

FIG. 3. Results of Applicants' studies to prove that Top2β is responsible for etoposide (VP-16)-induced DNA double-strand breaks.

FIG. 4. Results of Applicants' studies to prove that VP-16 induces fewer melanomas in the skin of skin-specific top2β knockout mice.

FIG. 5. Results of Applicants' studies to show that VP-16 poisons both Top2 isozymes equally but Top2β in the trapped Top2β complexes are preferential degraded to reveal the hidden DSBs.

FIG. 6. Results of Applicants' studies to show that dexrazoxane (ICRF-187) reduces doxorubicin-induced DNA damage.

FIG. 7. Results of Applicants' studies to show that doxorubicin-induced DNA damage is proteasome-dependent.

FIG. 8. Results of Applicants' studies to show that dexrazoxane induces proteasomal degradation of Top2β in H9C2 cardiomyocytes.

FIG. 9. Homology modeling of the N-terminal ATPase domain of human Top2α and Top2β in complex with dexrazoxane.

FIG. 10. Two proposed mechanisms for the antagonistic effect of dexrazoxane on doxorubicin-induced DNA damage.

### DETAILED DESCRIPTION OF THE PREFERRED EMBODIMENTS

Top2-targeting drugs (e.g. doxorubicin, epirubicin, mitoxantrone and etoposide) are the mainstay of chemotherapy. It is also known that there are two human Top2 isozymes, Top2α and Top2β. However, it has been unclear with respect to whether these two isozymes play different roles in tumor cell killing and in the development of secondary malignancies during the course of Top2-based chemotherapy.

Applicants have performed various studies that establish that the Top2α and Top2β isozymes have different roles in the development of secondary malignancies and tumor cell killing. The results of Applicants' studies suggest that the Top2β isozyme is primarily responsible for VP-16-induced carcinogenesis and also VP-16-induced DNA sequence rearrangements and double-strand breaks (DSBs). By contrast, Applicants also noted that VP-16 cytotoxicity in tumor cells appears to be primarily Top2α-dependent.

Applicants also evaluated whether Top2β-poisoning by current Top2 anticancer drugs leads to tissue toxicities. Applicants found that Top2β-targeting by doxorubicin in cardiomyocytes is responsible for DNA damage and cell death. Based on these results, Applicants discovered that Top2β-poisoning leads to tissue toxicities (e.g. cardiotoxicity) and thus is undesirable for Top2 anticancer drugs, and that it is highly desirable to develop Top2α preferential poisons.

Thus, one aspect of Applicants' invention provides a method for treating a patient with neoplasia comprising administering to the patient a therapeutically effective amount of a Top2α preferential poison. For the purpose of this Application, the term “Top2α preferential poison” means a Top2 poison that complexes the Top2α isozyme at least 10-fold as effectively as it complexes the Top2β isozyme as measured by in vitro DNA cleavage assay described in Bodley et al. Cancer Res. 49, 5969-5978 (1989). Accordingly, the term “Top2α preferential poison” also includes a Top2α-specific poison that has very little effect in poisoning the Top2β isozyme.

The therapeutic methods of the present invention can be used to treat a wide variety types of human neoplasias. Such neoplasias include but are not limited to leukemias, colorectal cancer, pancreatic cancer, lung cancer, prostate cancer, Wilms' tumor, neuroblastoma, soft tissue sarcoma, bone sarcoma, lymphoma, bladder cancer, breast cancer, stomach cancer, lung cancer, ovarian cancer, thyroid cancer, gastric cancer, testicular cancer, glioblastoma multiforme, Hodgkin's disease, Ewing's sarcoma, bronchogenic carcinoma and multiple myeloma.

Applicants have identified Top2α preferential poisons for use in connection with the present therapeutic methods. In certain embodiments, the Top2α preferential poisons include anthracyclines, ellipticines, acridines, carbolines, protoberberines, epipodophyllotoxicins, actinomycins, and their chemical analogs (i.e. their prodrugs, their metabolites, their protected derivates and their solvates).

In other preferred embodiments, the methods are practiced with Top2α preferential poisons selected from compounds of formula (I):

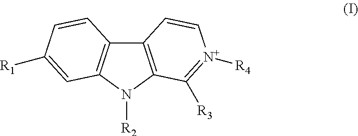

wherein

R₁ is H or —OR₅, wherein R₅ is (C₁₋₆)alkyl optionally substituted from by 1 to 5 radicals independently selected from a group of halo and halo-substituted (C₁₋₆)alkyl;

R₂ is H, —R₅, or (C₆₋₁₂)aryl(C₀₋₆)alkyl, wherein R₅ or (C₆₋₁₂)aryl(C₀₋₆)alkyl is optionally substituted from by 1 to 5 radicals independently selected from a group of halo, (C₁₋₆)alkyl, and halo-substituted (C₁₋₆)alkyl;

R₃ is H, (C₁₋₆)alkyl, or (C₆₋₁₂)aryl(C₀₋₆)alkyl, wherein (C₁₋₆)allyl or (C₆₋₁₂)aryl(C₀₋₆)alkyl is optionally substituted from by 1 to 5 radicals independently selected from a group of halo, (C₁₋₆)allyl, and halo-substituted (C₁₋₆)alkyl;

R₄ is H or (C₆₋₁₂)aryl(C₀₋₆)allyl optionally substituted from by 1 to 5 radicals independently selected from a group of halo, (C₁₋₆)allyl, and halo-substituted (C₁₋₆)alkyl.

When we refer to “C₀” (e.g., in “(C₆₋₁₂)aryl(C₀₋₆)alkyl”) we mean that the carbon or the alkyl group in the cited example does not exist.

In more preferred embodiments, the Top2α preferential poisons of formula (I), have R₁, R₂, R₃ and R₄ having definitions as follows: R₁ is H, C₄H₉O—, (CH₃)₂CHCH₂O— or (C₂H₅)₂CHO—; R₂ is —(CH₂)₃C₆H₅, C₂H₅—, —CH₂CH(CH₃)₂, —CH₂C₆H₄F, —CH₂C₆H₅, or —C₄H₉; R₃ is H, —CH₃, —C₆H₄Cl, or —CH₂C₆H₅; and R₄ is —CH₂C₆H₅, —CH₂C₆H₄Cl, —CH₂C₆H₄F, or —(CH₂)₃C₆H₅.

In certain preferred embodiments, the Top2α preferential poison of the present method is selected from a group of 2-benzyl-7-butoxy-9-isobutyl-1-methyl-9H-pyrido[3,4-b]indol-2-ium, 2-benzyl-7-isobutoxy-9-isobutyl-1-methyl-9H-pyrido[3,4-b]indol-2-ium, 2,9-dibenzyl-1-chlorophenyl-9H-pyrido[3,4-b]indol-2-ium, 1,2-dibenzyl-9-fluorobenzyl-9H-pyrido[3,4-b]indol-2-ium, and 9-butyl-1-chlorobenzyl-2-(3-phenylpropyl)-9H-pyrido[3,4-b]indol-2-ium.

Various β-carboline derivatives of compounds of Formula I above were evaluated for their antineoplastic activities and for their ability to preferentially poison Top2 isozymes. These compounds (i.e. those in Table 1 below) were also evaluated for Top2 isozyme-specific poisoning using the in vitro DNA cleavage assay described by Bodley et al. In the studies, purified recombinant human Top2 isozymes were used. The results of the evaluations are summarized in Table 2 below.

TABLE 1
Chemical structures of β-carbolines used in these examples:

| | | | | | IC₅₀ |
|---|---|---|---|---|---|
| Compound | R1 | R2 | R3 | R4 | (μM)* |
| # 1 | C4H9O | (CH2)3C6H5 | CH3 | CH2C6H5 | N.D.. |
| # 2 | C4H9O | C2H5 | CH3 | CH2C6H5 | N.D. |
| # 3 | C4H9O | CH2CH(CH3)2 | CH3 | CH2C6H5 | 4.6 |
| # 4 | (CH3)2CHCH2O | CH2CH(CH3)2 | CH3 | CH2C6H5 | 5.3 |
| # 5 | H | (CH2)3C6H5 | H | CH2C6H4Cl | 8.1 |
| # 6 | (C2H5)2CHO | (CH2)3C6H5 | CH3 | CH2C6H5 | 8.8 |
| # 7 | H | CH2C6H4F | C6H4Cl | CH2C6H4F | 4.4 |
| # 8 | H | CH2C6H5 | C6H4Cl | CH2C6H5 | 6.1 |
| # 9 | H | CH2C6H4F | CH2C6H5 | CH2C6H5 | 4.2 |
| # 10 | H | C4H9 | C6H4Cl | (CH2)3C6H5 | <1.7 |

*IC₅₀ was measured against A549 lung carcinoma
N.D. not determined.

TABLE 2
Poisoning activity against Top2α and Top2β:

| Compound | Top2α | Top2β |
|---|---|---|
| VP16 | ++++* | ++++ |
| # 1 | −** | − |
| # 2 | − | − |
| # 3 | ++ | − |
| # 4 | ++ | − |
| # 5 | − | − |
| # 6 | − | − |
| # 7 | − | − |
| # 8 | ++ | − |
| # 9 | ++ | − |
| # 10 | ++ | + |

*each + represent roughly 10-fold in activity
**non-detectable

The above studies have shown that VP-16 poisons Top2α and Top2β non-preferentially. In contrast, some of the cytotoxic β-carboline derivatives show preference for Top2α poisoning compared to Top2β poisoning. In particular, Compound Nos. 3, 4, 8 and 9 show roughly 20-fold in their activities against Top2α compared to their non-detectable activities against Top2β. (Table 2) and each of them still retains anti-neoplastic activity (Table 1). And Compound No. 10 roughly shows a 20-fold in its activity against Top2α compared to its 10-fold activity against the Top2β isozyme, and still possesses anti-neoplastic activity (Table 1).

The Top2 cleavage assay employed in the evaluation set forth in Table 2 was performed as described in Bodley et al. Cancer Res. 49, 5969-5978 (1989). Briefly, ³²P end-labeled linear DNA was incubated (at 37° C. for 30 min) with the purified recombinant human Top2α or Top2β (about 10 ng each) in a reaction containing 40 mM tris, pH8.0, 10 mM MgCl₂, 1 mM ATP, 100 mM KCl, 1 mM EDTA, 1 mM DTT, 30 μg/ml B SA and various concentrations of β-carbolines or VP-16. Reactions were terminated with SDS (final 1%) and digested with proteinase K (100 μg/ml at 50° C. for 1 hr). After adding gel loading solution, the reaction mixtures were analyzed by agarose gel electrophoresis. Gel was dried and exposed to x-ray films for visualization.

In human therapy, a dose of approximately 10-200 mg/m2 would be recommended for such Top2α preferential β-carboline poisons

In another aspect, the present invention is a method for treating a patient with neoplasia through a combination of administering a therapeutically effective amount of a Top2 inhibitor to the patient, followed by the administration of a therapeutically effective amount of a non-preferential Top2 poison (e.g., etoposide, doxorubicin, epirubicin or mitoxantrone), in which the non-preferential Top2 poison is administered at least 2 hours after the administration of the Top2 inhibitor. By “inhibitor,” we mean an agent that can stabilize the Top2 enzyme in a conformation that leads to enzyme degradation by proteases.

In certain embodiments, the therapeutic methods of the present invention are performed through: (a) administering to the patient a therapeutically effective amount of a Top2 inhibitor that preferentially inhibits Top2β isozyme over Top2α isozyme; (b) administrating a therapeutically effective amount of a non-preferential Top2 anticancer drug (“poison”) to the patient; wherein the Top2 inhibitor is administrated at least 2 hours prior to the administration of the Top2 poison.

In general, the therapeutic methods of this invention reduce or eliminate Top2β-damaging effects of non-preferential Top2 poisons by using the Top2 inhibitors, while preserving the efficacy of such poisons. It is contemplated that this pretreatment method can be practiced with existing non-preferential Top2 poisons, which can be administered in recommended dosages described in the 2008 Physician's Desk Reference, 62^{nd} Edition.

In certain embodiments of this invention the Top2 poisons used in the present methods include anthracyclines, ellipticines, acridines, carbolines, protoberberines, epipodophyllotoxicins, actinomycins, and their chemical analogs.

In certain embodiments, the Top2 inhibitors of the present methods are used to eliminate Top2β isozyme in target tissues. It is contemplated that all compounds that can induce Top2β degradation or elimination can be used in connection with the present methods. Additionally, compounds with enhanced selectivity toward Top2β (i.e. no or minimal activity toward Top2α) are expected to be better Top2 inhibitors that can be used to degrade Top2β without an effect on Top2α.

In certain preferred embodiments, compounds used as the Top2 inhibitors to induce Top2β degradation include ICRF-193, ICRF-187 (a/k/a dexrazoxane or Cardioxan), ICRF-154 and ICRF-159. These ICRF compounds are bis(2,6-dioxopiperazine) derivatives. It is also contemplated that prodrugs and metabolites of ICRF-193, ICRF-187, ICRF-154 and ICRF-159 can be used as Top2β inhibitors in this sense. Such Top2β inhibitors also include protected derivates and solvates of all these compounds.

Another aspect of this invention is a method for treating a patient with neoplasia comprising:co-administering a therapeutically effective amount of a Top2 poison and a therapeutically effective amount of a proteasome inhibitor to said patient.

Proteasome inhibitors include MG132 (i.e., N-[(Phenylmethoxy)carbonyl]-L-leucyl-N-[(1S)-1-formyl-3-methylbutyl]-L-leucinamide), bortezomib (Velcade), lactacystin, salinosporamide A, omuralide and NPI-0052 (as described in Cancer Cell, Volume 8, Issue 5, Pages 407-419 D. Chauhan). Preferably, the proteasome inhibitor comprises bortezomib.

In human therapy, a dose of approximately 0.5-20 mg/m² of MG132 or 0.1 to 5.0 mg/m² of bortezomib or 0.2 to 10 mg/m² of lactacystin would be recommended in this combination therapy method of this invention. A dose of approximately 0.1 to 10 mg/m² of the other proteasome inhibitors mentioned above would be recommended for such combination therapy. As to the co-administration embodiment, the Top2 poison can be administered in recommended dosages described in the 2008 Physician's Desk Reference, 62^{nd} Edition.

Proteasome inhibitors block transformation of Top2β cleavage complexes into DNA double-strand breaks (DSBs). Our studies demonstrated that a proteasome pathway is involved in the transformation of Top2β cleavage complexes (induced by non-preferential Top2-targeting anticancer drugs) into DNA double-strand breaks. Proteasome inhibitors can effectively block the formation of DSBs and thus prevent secondary malignancies and tissue toxicities associated with current Top2 drug-based therapy.

Another aspect of the present invention provides methods of identifying anti-neoplastic compounds, which preferentially poison the Top2α isozymes over the Top2β isozymes. Such methods can be practiced through screening of known Top2 poisons, their chemical analogs and also chemical libraries of compounds of other types.

In certain embodiments, the methods of identifying Top2α preferential poisons can be performed by: (a) evaluating the compound for its ability to poison Top2α isozyme; (b) evaluating the compound for its ability to poison Top2β isozyme; and (c) selecting a compound that preferentially poisons the Top2α isozyme over the Top2β isozyme. Further, methods can also be performed by evaluating a compound's ability to poison Top2β isozyme prior to the evaluation against the Top2α isozyme, which may eliminate the need to perform the latter evaluation if the former evaluation establishes that the compound has significant ability to poison Top2β isozyme.

In certain embodiments, the methods of identifying Top2α preferential poisons are practiced through high-throughput screening. In other certain embodiments, the methods of identifying Top2α preferential poisons include computer modeling and the use of structural activity relationship studies, either to be used alone, or in combination.

Yet in certain embodiments, the methods for identifying Top2α preferential poisons include using in vitro and/or in vivo Top2 isozyme-specific assays. In some preferred embodiments, the methods are performed by using multiple in vitro and in vivo Top2 isozyme-specific assays.

In certain preferred embodiments, the methods for identifying Top2α preferential poisons include using in vitro DNA cleavage assays. In the in vitro DNA cleavage assay, compounds as potential Top2 poisons are tested for their isozyme specificities through this assay using purified recombinant human Top2α (“hTop2α”) and human Top2β (“h Top2β”) isozymes. In this assay, the ability of various Top2 poisons to induce Top2 isozyme-mediated DNA cleavage of ³²P end-labeled linearized plasmid DNA was assessed. Thus, the relative specificity of various Top2 poisons against Top2 isozymes can be qualitatively determined based on the depletion of band intensities and/or the intensities of bands representing the cleavage products.

In some preferred embodiments, the methods for identifying Top2α preferential poisons also include using band depletion assays using tumor cells. This assay is used for further testing on Top2 isozyme-specific poisons identified by in vitro DNA cleavage assay by using breast cancer ZR-75-1 cells. In the assay, cells are treated with the test compound for a short time (e.g. typically 15-30 min) and then lysed with 1% SDS. Cell lysates will then be analyzed by immunoblotting using hTop2 isozyme-specific antibodies. Top2 isozyme-specific targeting is revealed by specific depletion of the immunoreactive bands corresponding to the two Top2 isozymes. For example, Top2α isozyme-specific compound is expected to specifically deplete the Top2α immunoband but not Top2β immunoband.

In certain preferred embodiments, the methods for identifying Top2α preferential poisons also include using in vivo Complex of Enzyme (ICE) assay. The ICE assay is quite sensitive to signals, since the assay is based on the increase of a signal from a low background.

Yet in another aspect, this invention also provides methods for identifying a Top2 inhibitor which preferentially inhibits the Top2β isozyme over the Top2α isozyme. In certain embodiments, the methods include high-throughput screening and specific inhibitor design. In certain preferred embodiments, the methods are practiced by using computer modeling and/or structural activity relationship studies. In certain embodiments, the methods are used to identify prodrugs and metabolites of the inhibitors.

The studies that form the foundation for this invention are summarized below and in the accompanying figures and examples described below.

### Example 1

### Materials and Methods

Mouse Strains. Skin-specific top2β knockout mice and their TOP2β⁺ controls were derived from the top2β^{+/flox2} and top2β^{flox2/flox2} lines previously reported. The top2β^{flox2} allele contains two loxP sites flanking three Top2β exons that encode a region of TopIIβ containing the active-site tyrosyl residue; this allele expresses wild-type TopIIβ, but is converted to a null allele top2β^{Δ2} upon exposure to Cre recombinase. Skin-specific deletion within the floxed top2β allele is achieved by crossing the top2β^{flox2} lines with mice expressing Cre recombinase from the keratin 14 promoter (kindly provided by Andrew P. McMahon, Harvard University). The K14-Cre transgenic mouse line expresses Cre in the keratinocytes of the epidermis and the hair follicles during prenatal and postnatal development. Mice with the genotype K14-Cre top2β^{flox2/flox2}, K14-Cre top2β^{+/flox2}, top2β^{flox2/flox2} and top2β^{+/flox2} were generated and used in this study; with the exception of K14-Cre top2β^{flox2/flox2} mice, which lack TopIIβ in skin cells, all the others are phenotypically Top2β⁺ in all tissues. The K14-Cre top2β^{flox2/flox2} skin-specific top2β knockout mice exhibit a normal lifespan and show no skin abnormality other than cyclic alopecia; detailed description of these mice will be reported elsewhere.

Genotyping. Mouse genomic DNA samples were isolated from mouse tails using the DNeasy Blood & Tissue Kit (Qiagen). The appearance of the top2β^{flox2} and top2β^{Δ2} alleles was confirmed by PCR analysis of the samples using respectively primer pairs PR2 (5′-TCATTGGGAGGCCAGAGCATC-3′) and PR3 (5′-ATATGGTACAGCAACAAAGCATTTGACATA-3′), and PR3 and PR7 (5′-GAATTGTTTGCTGTGGATGCATGTA-3′). PCR reactions using primers PR2 and PR3 were also used to generate a unique fragment from the wild-type Top2β⁺ allele. The presence of the K14-Cre allele was confirmed by PCR analysis using primers K14CreR (5′-TTCCTCAGGAGTGTCTTCGC-3′) and K14CreF (5′-GTCCATGTCCTTCCTGAAGC-3′).

Carcinogenesis Assay Using a Mouse Skin Model. Seven-week old skin-specific top2β knockout mice and their TOP2β⁺ controls were used in the study. The back of each mouse was shaved two days prior to treatment. The tumor initiator DMBA (1 μmol in 100 μl of DMSO) was applied once in the first week, followed by various treatments (two applications per week) for six groups of animals: Group 1, DMSO (100 μl), 5 applications; Group 2, VP-16 (10 μmol in 100 μl DMSO), 5 applications; Group 3, VP-16 (20 μmol in 200 μl of DMSO), 3 applications; Group 4, the tumor promoter TPA (phorbol 12-tetradecanoate 13-acetate, 17 nmol in 100 μl DMSO), 8 applications; Group 5, VP-16 (5 mmol in 100 μl DMSO), 5 application; Group 6, VP-16 (5 μmol in 100 μl DMSO), 10 applications. Mice were examined every week for appearance of melanomas on their skins. The number of melanomas visibly notable was scored at the end of the 16^{th} week. The average numbers of tumors induced in different treatment groups were compared using Student's t-test.

Histochemical and Immunohistochemical Analyses. Skin samples were dissected from euthanized mice and processed for embedding in the OCT compound (Tissue Tek). For cryopreservation, samples were fixed in 4% paraformaldehyde in 1×PBS for 3 hr at 4° C. After washing with ice-cold PBS for 30 min, samples were immersed overnight in 30% sucrose solution in PBS at 4° C., followed by embedding in OCT. For immunohistochemical analysis, cryosections (8-10 μm thick) were fixed in 4% paraformaldehyde for 10 min at room temperature, and washed four times in PBS (2 min each). Antigen retrieval was performed by incubation in 1% SDS at room temperature for 5 min, followed by washing four times in PBS (2 min each). For melanin bleaching, tissue sections were exposed to potassium permanganate (2.5 g/l) for 10 min and then oxalic acid (5 g/l) for 3 min at room temperature. After washing in PBS, sections were incubated in ADB solution (0.05% Triton X-100, 10% goat serum and 3% BSA in PBS) for 30 min. Mouse melanoma cocktail antibody (1:100 dilution, Abcam) or rabbit anti-TopIIIβ antibody (1:100 dilution, Santa Cruz) was applied to skin sections and incubated overnight in a humidified chamber at 4° C. After four washes (5 min each) in TBST (Tris-buffered saline, 0.1% Tween 20), skin sections were incubated with Cy3- or Cy2-conjugated secondary antibodies (Jackson ImmunoResearch) for 30 min at 37° C. After washing in TBST (four times, 5 min each), slides were mounted with Gel/Mount (Biomeda Corp.). Images were visualized under a fluorescence microscope and photographed with a charge-coupled-device (CCD) camera.

Cells. Primary MEFs were isolated from E13.5 Top2β^{+/+}, Top2β^{+/Δ2} and Top2β^{Δ2/Δ2} mouse embryos, as described (the Top2β^{Δ2} allele contains a deletion in the coding region of the active-site tyrosyl residue). SV40-transformed MEFs were obtained by transformation with pAN2 DNA as described. Cells were maintained in DMEM supplemented with 10% FetalPlex animal serum complex (Gemini Bio-Products), L-glutamine (2 mM), penicillin (100 units/ml), and streptomycin (100 μg/ml) in a 37° C. incubator with 5% CO₂. PC12 cells were first clonally selected and then used to generate Top2β-shRNA and control-shRNA knockdown cells. A rat Top2β-shRNA sequence (5′-GCCCCCGTTATATCTTCAC-3′) was generated based on the 643-bp partial rat TopIIβ cDNA sequence (GenBank™ accession number D14046). Duplex (5′-TGCCCCCGTTATATCTTCACTTCAAGAGAGTGAAGATATAACGGGGGCTTTTTC-3′) DNA was made and cloned into the LentiLox 3.7 vector (obtained from Dr. van Parijs, MIT). The control-shRNA sequence (5′-GCGCGCGTTAAATCTTCAC-3′) was created by altering three nucleotides in the rat Top2β-shRNA sequence (underlined). The duplex (5′-TGCGCGCGTTAAATCTTCACTTCAAGAGAGTGAAGATTTAACGCGCGCTTTITC-3′) DNA was cloned into the LentiLox 3.7 vector. The shRNA expressing LentiLox 3.7 vectors were then inserted with the PGK-driven Ned gene. Lentiviral stock was prepared and virus-infected PC12 cells were selected from a two week culture in the presence of 700 μg/ml G418. Single colonies were isolated and characterized, and cultured in a 37° C. incubator with 5% CO₂, in RPMI 1640 medium supplemented with 10% horse serum, 5% FetalPlex animal serum complex, L-glutamine (2 mM), penicillin (100 units/ml), and streptomycin (100 μg/ml), in flasks coated with collagen type I (BD Biosciences, Bedford, Mass.).

Plasmid Integration Assay. Transformed top2β^{+/Δ2} and top2β^{Δ2/Δ2} MEFs were plated in six-well plates (4×10⁵ cells per well) one day prior to transfection. Transfection was performed with EcoRI-linearized pUCSV-BSD plasmid (containing the blasticidin resistance gene) using the Cellfectin (Invitrogen) transfection reagent (0.1 μg DNA+2 μl Cellfectin). VP-16 was added at the time of transfection. After 6 hr, cells were washed and trypsinized. A small aliquot was removed, reseeded into fresh medium and grown without the selection agent for survival determination. The rest of the cells were reseeded into fresh medium in a 10 cm plate. After 24 hr, the selection agent blasticidin (3 μg/ml) was added. Colonies were stained with methylene blue and counted after 10 days. Where indicated, the proteasome inhibitor MG132 (2 μM) was added 30 min prior to and during transfection. Integration frequency was determined as the ratio of the number of blasticidin-resistant colonies and the number of surviving cells.

### Example 2

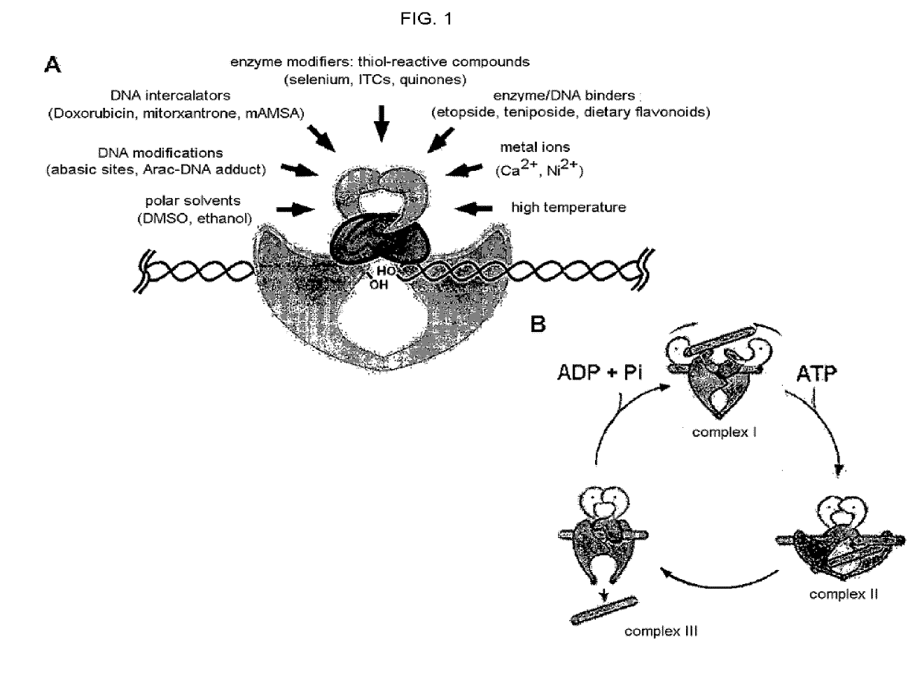

FIG. 1. The Top2 cleavage complex and the catalytic cycle. A. Stabilization of Top2 cleavage complexes by various agents and stress conditions. B. Catalytic reaction of Top2. DNA G-segment and T-segment are represented by two rods. The N-terminal ATPase domains of the Top2 homodimer are drawn as a pair of jaws with ATP binding sites marked by *. There are two classes of Top2 inhibitors, those which trap Top2 covalent reaction intermediate (e.g. non-preferential Top2poisons such as etoposide and doxorubicin) and those which inhibit the ATPase activity (e.g. bisdioxopiperazines such as ICRF-193 and ICRF-187). The ICRF-187 (dexrazoxane) binding site is also in the ATPase domain near the ATP binding site.

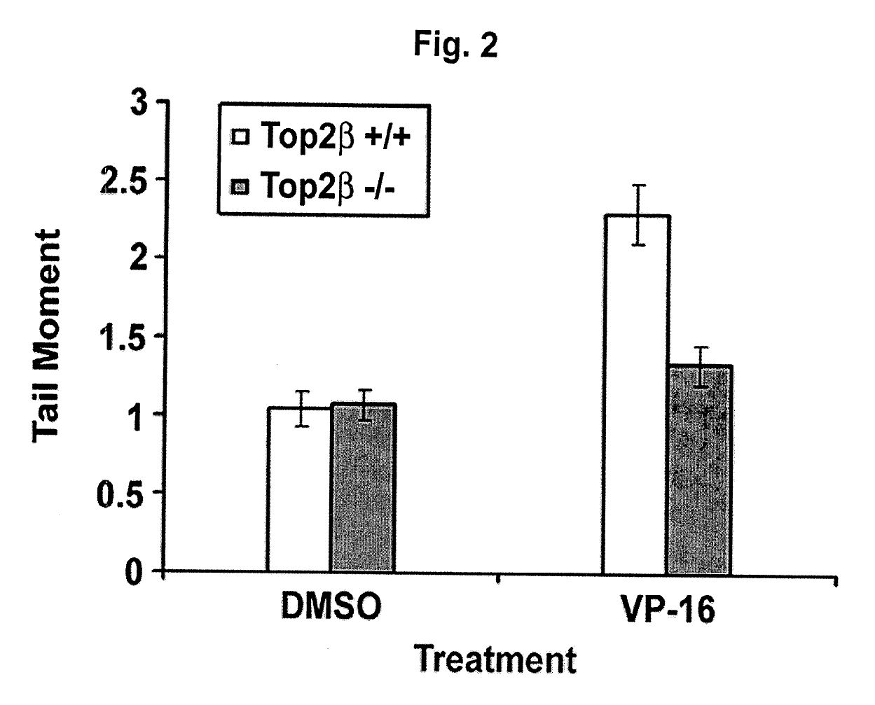

FIG. 2 Top2β is responsible for etoposide (VP-16)-induced DNA double-strand breaks. Both wild type and Top2β knockout mouse embryonic fibroblasts (MEFs) were treated with either DMSO (solvent control) or VP-16 (250 μM) for 90 min, followed by incubated in drug-free media for 30 min. Neutral comet assay was then performed. Tail moment (average from 100 cells) was determined for each treatment.

### Example 3

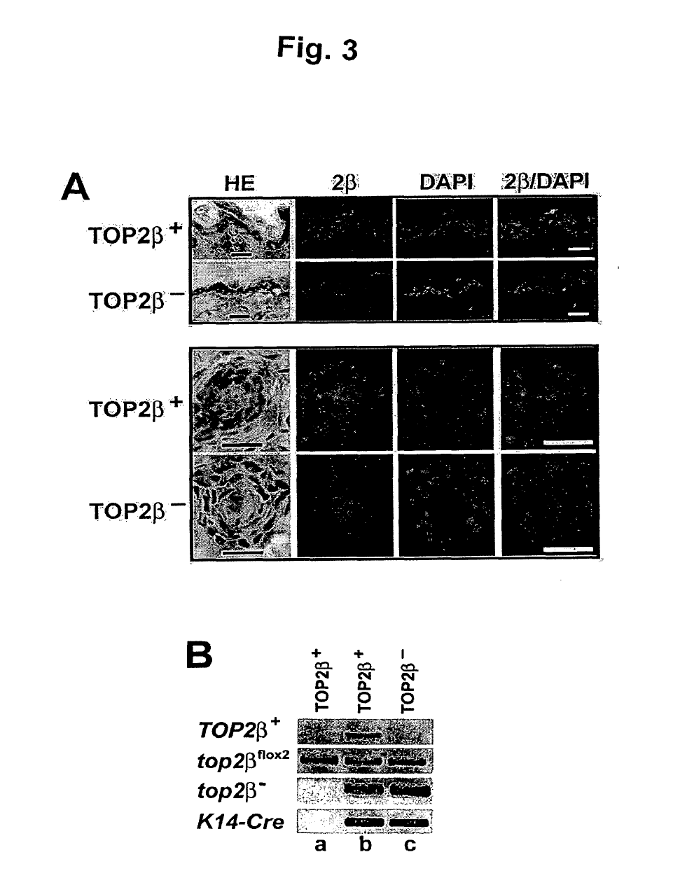

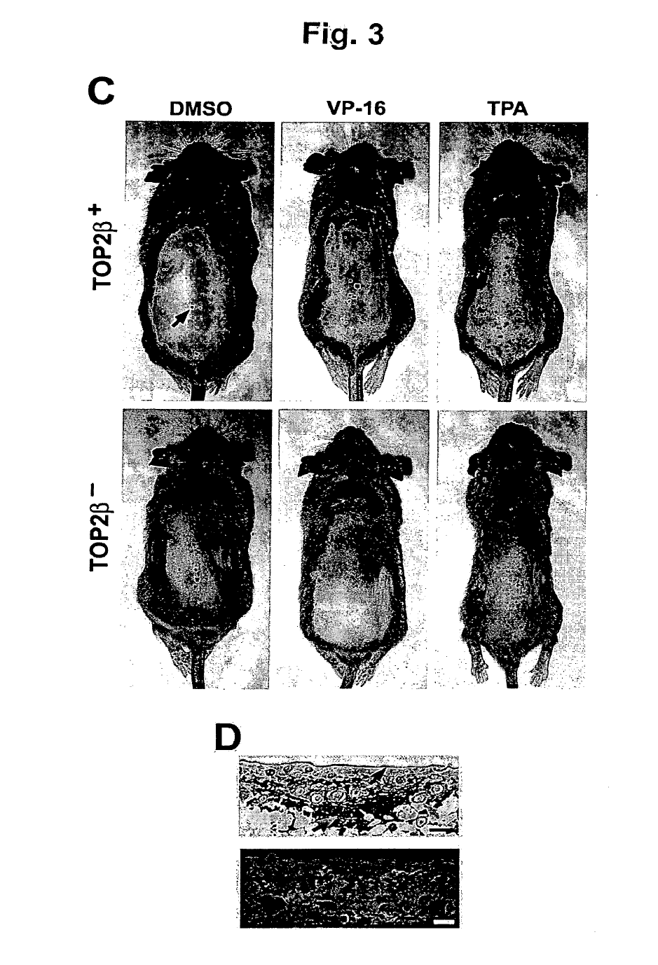

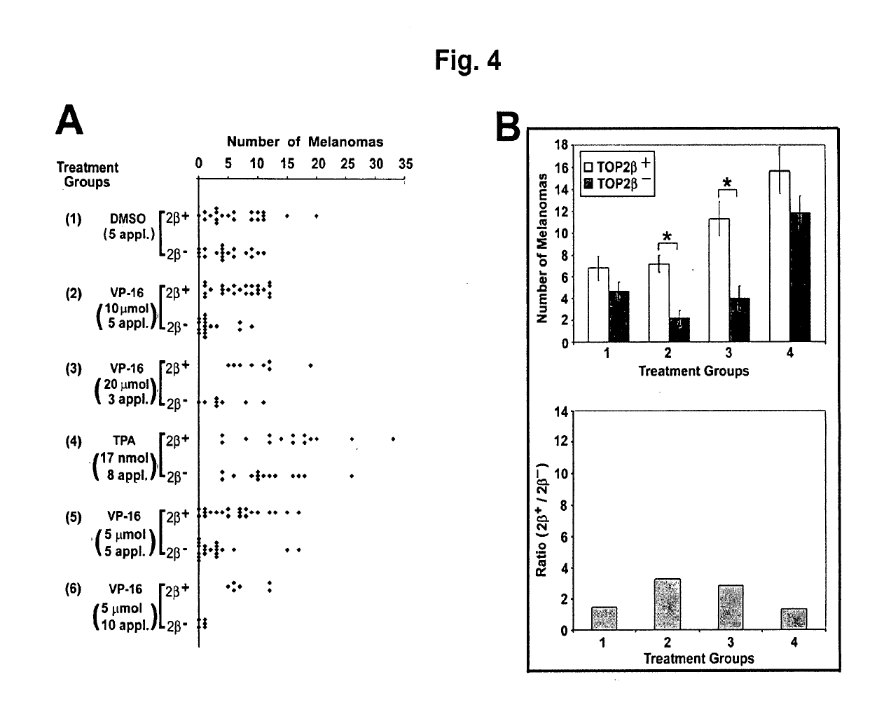

FIG. 3 VP-16 induces melanomas in the skin of DMBA-initiated mice. (A) Absence of Top2β in the epidermis and hair follicles of skin-specific top2β knockout mice (TOP2β⁻). 8-10 μm thick cryosections of the skin of TOP2β⁺ and TOP2β⁻ mice (epidermis, upper panel; hair follicle, lower panel) were stained with hematoxylin and eosin (labeled HE, first column) or anti-Top2β antibody (labeled 2β, second column)/DAPI (third column). The merged images of 2β- and DAPI-stained sections are shown in the fourth column (labeled IIβ/DAPI). (B) PCR-based genotyping of TOP2β⁺ and TOP2β⁻ mice. Genomic DNA samples from mouse tails were genotyped by PCR, using respective primer sets specific to the Top2β⁺, top2β^{flox2}, top2β⁻ or K14-Cre alleles as described in Materials and Methods. Examples of genotyping results are shown here; Top2β^{+/flox2} (lane 1), K14-Cre top2β^{+/flox2} (lane 2), and K14-Cre top2β^{flox2/flox2} (lane 3). Skin cells of K14-Cre top2β^{flox2/flox2} are phenotypically TOP2β⁻, and cells from Top2β^{+/flox2} and K14-Cre top2β^{+/flox2} mice are TOP2β⁺. (C) VP-16-induced melanomas in the skin of TOP2β⁺ and skin-specific Top2β knockout mice (TOP2β⁻). Representative pictures of DMBA-initiated mice treated with DMSO (vehicle control), VP-16 or TPA are shown. The blue arrow points to a melanoma. (D) Histological and immunohistochemical analyses of melanomas in the mouse skin. Consecutive sections of skin melanomas were stained with either HE or melanoma-specific antibodies. Representative pictures of HE staining (upper panel) and melanoma antibody staining (lower panel) are shown. The arrow points to the melanoma mass, the blue arrow the epidermis, and the green arrow a hair follicle. Scale bars: 10 μm in (A) and 100 μm in (D).

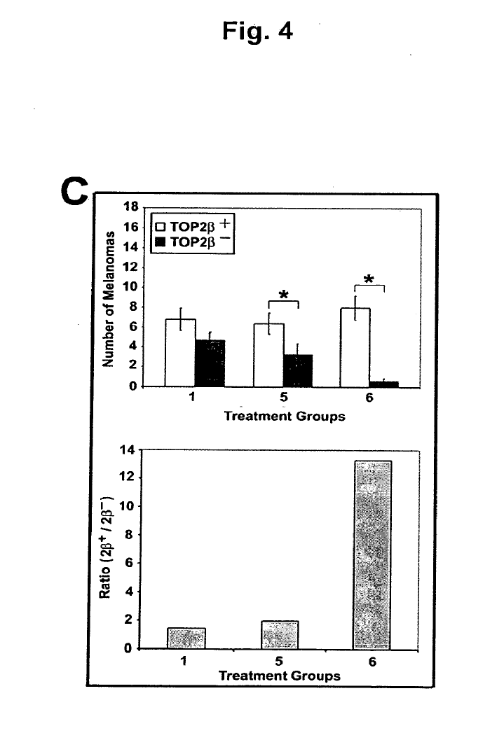

FIG. 4 VP-16 induces fewer melanomas in the skin of skin-specific top2β knockout mice. (A) The number of melanomas in the skin of each mouse of a specific treatment group is plotted. The symbol “2β⁺” denotes Top2β⁺ mice, and “2β⁻” denotes skin-specific Top2β knockout (Top2β⁻) mice. The six treatment groups (see the numbers in parenthesis) are shown at the bottom of the graph, together with their treatment descriptions. (B) The average number of melanomas per mouse for each treatment group is plotted. The treatment groups are labeled Group 1, Group 2, Group 3 and Group 4. (C) The same as in B except that results from the treatment groups 1, 5 and 6 are plotted. The difference in the average number of melanomas per mouse between Top2β⁺ and Top2β⁻ mice is statistically significant (p<0.05) for groups 2, 3, 5 and 6 (marked with *).

The results shown by FIGS. 3 and 4: The studies show that VP-16-induced melanomas in the skin of DMBA-initiated mice are Top2β-dependent. In order to evaluate the role of Top2β in VP-16-induced carcinogenesis, skin-specific top2β knockout mice (K14-Cre Top2β^{flox2/flox2}) and Top2β⁺ controls (Top2β^{flox2/flox2}, Top2β^{+/flox2}, and K14-Cre top2β^{+/flox2}) were generated (see FIG. 3B for genotyping examples). As shown in FIG. 3A, Top2β is absent in both the epidermis (upper panel) and hair follicles (lower panel) of K14-Cre top2β^{flox2/flox2} mice, to be referred to hereafter as the TOP2β⁻ mice, as evidenced by the absence of Top2β immunostaining in DAPI-positive nuclei. In addition, Cre-mediated deletion of the foxed Top2β locus is evidenced by the appearance of the PCR product corresponding to the Top2β^{Δ2} allele, to be referred to hereafter as the Top2β allele (see lanes 2 and 3 in FIG. 3B). Age-matched 7 week-old mice were used for skin carcinogenesis studies. Both Top2β⁺ and Top2β⁻ mice were initiated with a single application of DMBA, followed by various treatments (see the six treatment groups in Materials and Methods). Under the treatment conditions, these mice developed skin melanomas (see representative pictures in FIG. 3C of mice with skin melanomas from different treatment groups). Histology of a typical melanoma in the mouse skin is shown in FIG. 3D (upper panel). The expansive dark brown area, showing aggregation of pigmented cells (melanin expressing melanocytes), is indicative of melanoma. Immunohistochemical analysis of the tumor with mouse melanoma cocktail antibody also confirmed the presence of melanoma (FIG. 3D, lower panel).

### Example 4

The number of melanomas in the skin of each mouse in various treatment groups was recorded and all data are summarized in FIG. 4A. The average number of melanomas per mouse in different treatment groups is also plotted for each treatment group (FIGS. 4B and 4C). As shown in FIG. 4B (unfilled bars), VP-16 treatment of DMBA-initiated Top2β⁺ mice (see Groups 2 and 3 for 10 μmol×5 applications and 20 μmol×3 applications of VP-16, respectively) show an increase in the average number of melanomas per mouse (by about 10% and 60%, respectively) when compared to treatment with DMSO alone (Group 1). Surprisingly, VP-16 treatment of DMBA-initiated Top2β⁻ mice decreases, rather than increases, the average number of melanomas per mouse, by ˜50 and 15% respectively in Groups 2 and 3 relative to the Group 1 controls treated with DMSO alone (FIG. 4B, filled bars). This decrease probably reflects a combination of two factors: the absence of VP-16-induced melanomas owing to the absence of Top2β, and the antitumor activity of VP-16 (which is largely Top2α-dependent, to be discussed later).

Thus, increasing the number of VP-16 applications would be expected to further reduce the number of melanomas in Top2β⁻ mice. Indeed, as shown in FIG. 4C (filled bars), increasing the number of VP-16 applications (5 mol/application) from 5 (Group 5) to 10 (Group 6) significantly decreases the average number of melanomas in the skin of TOP2β⁻ mice, by ˜30% and 87% respectively relative to DMSO treatment alone (Group 1). As a positive control, DMBA-initiated TOP2β⁺ and TOP2β⁻ mice were also treated with TPA (see FIG. 4B, Group 4). Accordingly, TPA treatment of the TOP2β⁺ mice greatly stimulated the average number of melanomas per mouse (by 130%) relative to DMSO treatment (FIG. 4B, unfilled bars). However, in contrast to VP-16 treatment, exposure to TPA causes a similar degree of increase (150%) in skin melanoma in TOP2β⁻ mice (FIG. 4B, filled bars).

The effect of Top2β on the number of VP-16-induced melanomas in mouse skin is more evident by examine the ratio of the average number of melanomas per mouse in Top2β⁺ versus that in Top2β⁻ mice. For the VP-16-treated groups, the ratios are 2.0 (Group 5), 2.8 (Group 3), 3.3 (Group 2) and 13 (Group 6). By contrast, the ratios are 1.5 and 1.3, respectively, for Groups 1 (vehicle control) and 4 (TPA treatment). The differences in the number of VP-16-induced melanomas in Top2β⁺ and Top2β⁻ mice are statistically significant (p<0.05, see groups marked by * in FIGS. 4B and 4C). These results demonstrate that VP-16 but not TPA-promoted melanomas in the mouse skin are primarily Top2β-mediated.

### Example 5

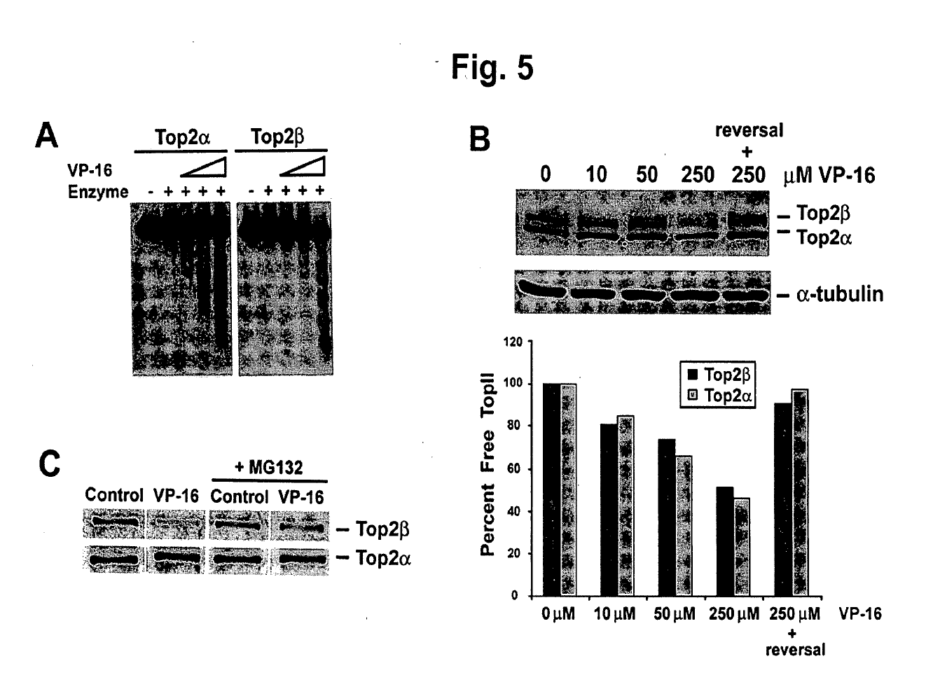

FIG. 5 VP-16 poisons both Top2 isozymes equally but Top2β in the trapped Top2β complexes are preferential degraded to reveal the hidden DSBs. (A) VP-16 poisons Top2 isozymes equally in vitro. Cleavage assays were performed. VP-16 concentrations used were 2.0, 20 and 200 μM. (B) VP-16 effectively traps both Top2α and Top2β cleavage complexes in vivo. Transformed Top2β^{+/+} MEFs were treated with VP-16 (0, 10, 50 and 250 μM) for 15 min and the amounts of Top2 (2α and 2β) cleavage complexes were measured by the band depletion assay. The results are quantified and the percent free Top2 is plotted for each treatment (lower panel). VP-16-induced Top2 cleavage complexes are also reversed by a further incubation in VP-16-free medium for 50 min (last lane, labeled reversal+250 μM VP-16). (C) VP-16 induces preferential down-regulation of Top2β. Transformed Top2β^{+/+} MEFs were treated with VP-16 (50 μM, 2 hr) in the presence or absence of the proteasome inhibitor, MG132 (2 μM). The cleavage complexes in treated cells were reversed by an additional incubation at 37° C. for 30 min, following by alkaline lysis and S7 nuclease treatment. The amounts of Top2 isozymes were measured by Western blotting.

Studies have demonstrated that in VP-16-treated cells the trapped covalent complexes of the Top2β isozyme are preferentially degraded over the Top2α complexes through a proteasome-dependent pathway, and suggested that preferential proteasomal degradation of VP-16-induced Top2β cleavage complexes leads to DSB formation. In support of this notion, co-treatment with MG132 is found to abolish VP-16-induced DSBs, as evidenced by neutral comet assays (data not shown). Thus it appears likely that the preferential role of the Top2β isozyme in VP-16-induced DSBs and DNA sequence rearrangements is due to the greater sensitivity of Top2β cleavage complexes to proteasome-mediated degradation. Indeed, Top2β is found to be preferentially degraded over Top2α in SV40-transformed Top2α^{+/+} Top2β^{+/+} MEFs treated with VP-16 (FIG. 5C).

Top2β contributes minimally to VP-16 cytotoxicity in transformed cells. The above studies suggest that Top2β is primarily responsible for VP-16-induced DSBs and DNA sequence rearrangements. To test if Top2β is also important for VP-16 cytotoxicity, we determined the IC₅₀ of VP-16 in two pairs of transformed Top2β knockout/knockdown cells using 4-day MTT assay (in triplicates). The IC₅₀ values of VP-16 (0.038±0.007 vs. 0.040±0.006 μM) are the same (p=0.43, t-test) for Top2β^{+/-} and Top2β^{-/-} MEFs. The IC₅₀ values of VP-16 (1.9±0.1 vs. 1.6±0.1 μM) are also about the same (p=0.19, t-test) for control-shRNA-PC12 and Top2β-snRNA-PC12 cells (Top2β protein is reduced about 90% in Top2β-shRNA-PC12 cells). These results suggest that in terms of VP-16 cytotoxicity in transformed cells, it is Top2α, and not Top2β, that plays a major role. Thus, the two Top2 isozymes appear to play very different roles in VP-16-promoted carcinogenesis and tumor cell killing.

Previous studies have also demonstrated that VP-16 induces papillomas on the skin of DMBA-initiated mice in a classical two-stage carcinogenesis model; furthermore, switching the order of VP-16 and DMBA applications has no effect on the incidence of papillomas, indicating that the drug behaves as a stage I (convertogenic) tumor promoter. The convertogenic activity of VP-16 has been attributed to its induction of DNA sequence rearrangements. In the present study, we have used skin-specific Top2β knockout mice to evaluate the roles of the two isozymes of DNA Top2, Top2α and Top2β in VP-16-induced carcinogenesis. Melanomas rather than papillomas are the main tumor type detected in the mouse skin in our studies, plausibly due to genetic background differences of the mouse strains used in these studies: Previous studies employed albino mouse strains that probably produce no visible melanoma because of a lack of melanin expression in the skin of these mice, while the mice used in the present studies have a mix genetic background including 129SvEv (at least 75%), various degrees of Balb/c, and C57/BL6, and express melanin in their skin. VP-16 is shown to induce 2- to 13-fold more melanomas, depending on the dose and schedule of VP-16 treatment, in the skin of DMBA-initiated TOP2β⁺ mice than in the skin of similarly treated skin-specific Top2β knockout mice. By contrast, the classical tumor promoter, TPA, induced about the same number of skin melanomas in DMBA-initiated mice whether Top2β is expressed in the skin or not. These results suggest that it is the Top2β isozyme that plays a predominant role in VP-16-induced carcinogenesis.

The above conclusion is further supported by studies in tissue culture models. Using a plasmid integration assay to monitor DNA sequence rearrangements, VP-16-stimulated plasmid integration is shown to be Top2β-dependent: stimulation of integration frequency by VP-16 is much more significant in SV40-transformed MEFs derived from top2β^{+/-} mice, which express Top2β, than SV40-transformed MEFs derived from top2β^{-/-} mice, which do not. Furthermore, the proteasome inhibitor MG132 blocks VP-16-stimulated plasmid integration, suggesting that VP-16-induced DNA sequence rearrangements involve the proteasome pathway. This result is consistent with that of a recent study implicating the involvement of the proteasome pathway in processing VP-16-induced TopIIβ-DNA covalent complexes into DSBs.

The predominant role of the Top2β isozyme in mediating VP-16-induced carcinogenesis and DNA sequence rearrangements can be attributed to the propensity of the Top2β isozyme in DSB formation upon VP-16 treatment. Neutral comet assay indicates that VP-16-induced DSBs are Top2β-dependent in both primary and SV40-transformed MEFs. Furthermore, the predominant role of the Top2β isozyme in VP-16 mediated DSB formation is likely the result of a greater sensitivity of the DNA cleavage complexes of Top2β, relative to the DNA cleavage complexes of Top2α, in proteasome-mediated degradation. Whereas the two isozymes exhibit comparable propensities in VP-16 induced covalent complex formation, the Top2β-concealed DNA breaks in the covalent complexes appear to be more easily converted to DSBs by the proteasome degradation pathway.

Based on these and other results, a model for the role of Top2β in VP-16-induced DSBs, DNA sequence rearrangements and carcinogenesis is proposed. In this model, VP-16 stabilizes reversible Top2β cleavage complexes. These Top2β cleavage complexes are converted into non-reversible Top2β-DNA covalent complexes in part through transcriptional collisions. Top2β-DNA covalent complexes then undergo proteasomal degradation, leading to the exposure of the hidden DSBs in them. Subsequent processing of these DSBs through non-homologous end-joining (NHEJ) may lead to DNA sequence rearrangements and carcinogenesis. It is unclear why the DNA cleavage complexes of Top2β are more sensitive to proteasome-mediated degradation than their Top2α counterparts. Because proteasomal degradation of Top2 cleavage complexes is partially transcription-dependent, however, the preferential sensitivity of the Top2β complexes to proteasomal degradation might be related to the preferential involvement of Top2β in transcription. Further studies are necessary to establish the molecular pathways in processing the Top2-DNA covalent complexes.

Whereas Top23 rather than Top2α is shown to have a predominant role in VP-16-induced carcinogenesis, our studies of Top2β knockout and knockdown cells suggest that the opposite is the case in VP-16 cytotoxicity against transformed cells. The importance of Top2α in VP-16 cytotoxicity is consistent with results from the previous studies that the Top2α gene is mutated in cell lines selected for lower levels of resistance to non-preferential Top2 drugs, and the Top2β gene is mutated only in Top2α mutant cells selected for higher levels of resistance to Top2 drugs. It has been suggested that the collision between the replication forks and Top2 cleavage complexes plays a major role in VP-16 cytotoxicity. Consequently, the predominant role of Top2α in DNA replication may lead to more frequent collisions with the replication forks and thus cytotoxicity.

### Example 6

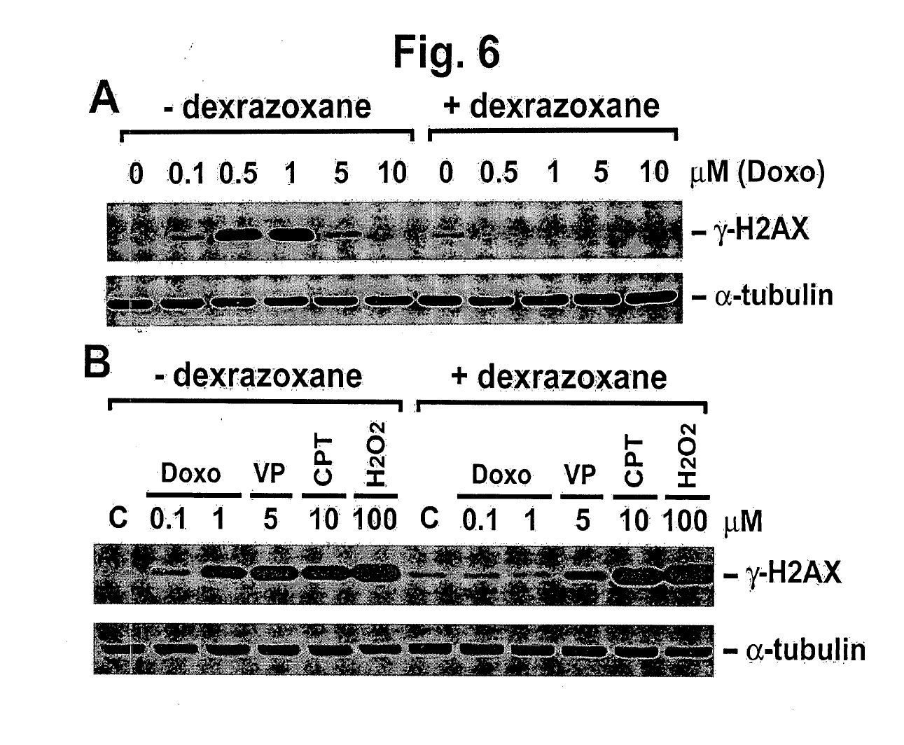

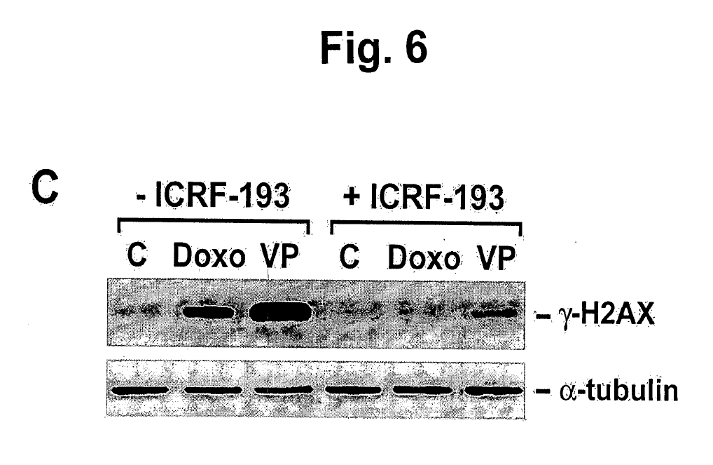

FIG. 6 Dexrazoxane reduces doxorubicin-induced DNA damage. A, 1.5×10⁵ H9C2 cardiomyocytes were treated with 0, 0.1, 0.5, 1, 5 and 10 μM doxorubicin (Doxo) in the absence (labeled −dexrazoxane) or presence of dexrazoxane (200 μM, labeled +dexrazoxane) for 1 hr. Cell lysates were analyzed by Western blotting using anti-γ-H2AX or anti-α-tubulin antibody (for assessing protein loading). B, H9C2 cardiomyocytes were treated with 0.1% DMSO (labeled C, for solvent control), 0.1 or 1 μM doxorubicin (Doxo), 5 μM VP-16 (VP), 10 μM CPT or 100 μM H₂O₂, in the absence (labeled −dexrazoxane) or presence (labeled +dexrazoxane) of dexrazoxane (200 μM) for 1 hr. Cells were then lysed and analyzed by Western blotting using anti-γ-H2AX or anti-α-tubulin antibody. C, H9C2 cardiomyocytes were treated with 0.1% DMSO (labeled C, for solvent control), 0.5 μM doxorubicin (labeled Doxo) or 10 μM VP-16 (labeled VP) in the absence (labeled −ICRF-193) or presence (labeled +ICRF-193) of ICRF-193 (50 μM) for 1 hr. Cells were then lysed and analyzed by Western blotting using anti-γ-H2AX or anti-α-tubulin antibody.

As shown in FIG. 6A, doxorubicin induced the DNA damage signal, γ-H2AX (Ser-139-phosphorylated H2AX, a key DNA damage signal induced by DNA double-strand breaks), in H9C2 cardiomyocytes. Doxorubicin-induced γ-H2AX was concentration-dependent up to 1 μM. At higher concentrations of doxorubicin (5 and 10 μM), the γ-H2AX signal was dramatically reduced. This pattern of concentration-dependent inhibition is reminiscent of dose-dependent inhibition of doxorubicin-induced Top2 cleavable/cleavage complexes. In the presence of dexrazoxane (200 μM), the doxorubicin-induced γ-H2AX signal was completely blocked. This blocking effect appears to be specific to Top2-directed drugs such as doxorubicin and VP-16 (FIG. 6B).

To test whether the blocking effect of dexrazoxane was due to inhibition of Top2, another well characterized Top2 catalytic inhibitor, ICRF-193, was also tested. As shown in FIG. 5C, both the doxorubicin (0.5 μM, labeled Doxo)- and the VP-16 (10 μM, labeled VP)-induced DNA damage signal, γ-H2AX, was indeed abolished by co-treatment with ICRF-193 (FIG. 6C).

### Example 7

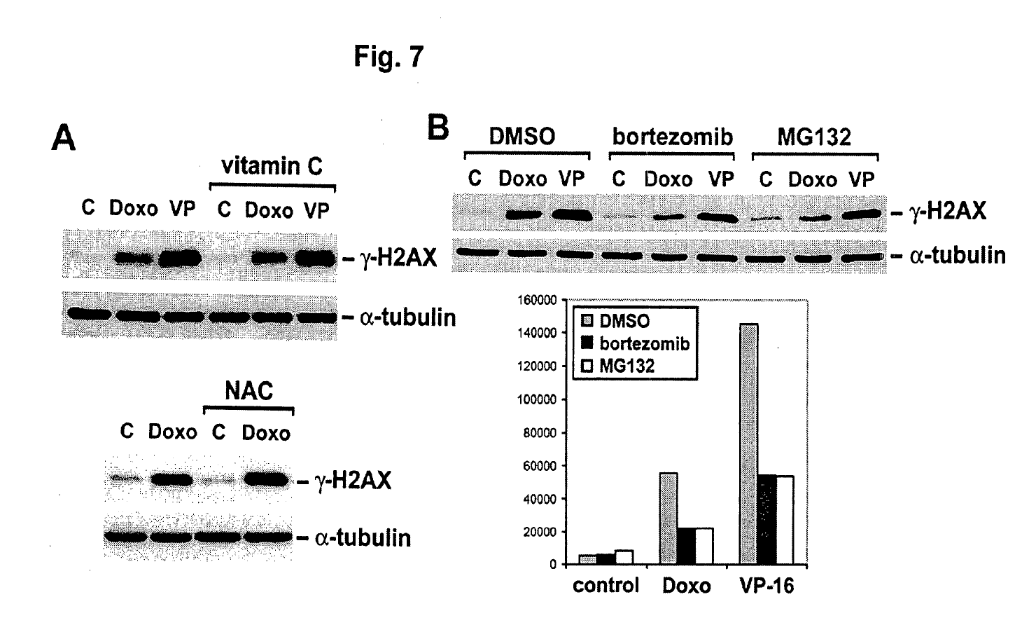

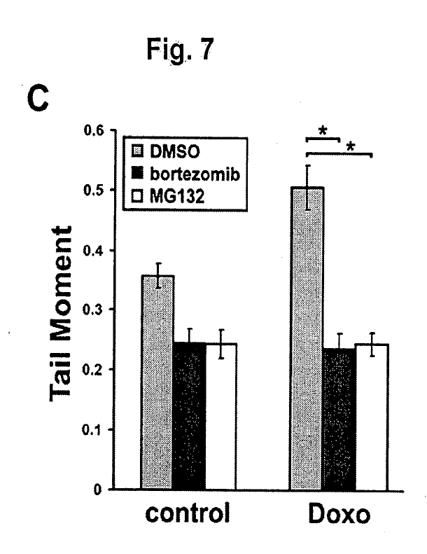

FIG. 7 Doxorubicin-induced DNA damage is proteasome-dependent. A, H9C2 cardiomyocytes were treated with 0.1% DMSO (labeled C, for solvent control), 0.5 μM doxorubicin (labeled Doxo) or 10 μM VP-16 (labeled VP) for 1 hr in the presence or absence of 100 μg/ml vitamin C (upper panel) or 100 μg/ml N-Acetyl Cysteine (labeled NAC) (lower panel). Vitamin C and NAC were added 30 min prior to doxorubicin. Cell lysates were then analyzed by Western blotting using anti-γ-H2AX or anti-α-tubulin antibody. B, 1.5×10⁵ H9C2 cardiomyocytes were treated with 0.1% DMSO (labeled C, solvent control), doxorubicin (0.5 μM, labeled Doxo) or VP-16 (10 μM, labeled VP) for 1 hr, in the absence or presence of either bortezomib (1 μM) or MG132 (4 μM). Bortezomib and MG132 were added 30 min prior to doxorubicin or VP-16. Cell lysates were then analyzed by Western blotting (upper panel) using anti-γ-H2AX or anti-α-tubulin antibody. Quantification of γ-H2AX signals is shown in the lower panel. C, H9C2 cells were treated with 0.1% DMSO (labeled DMSO), bortezomib (1 μM) or MG132 (4 μM) for 30 min, followed by co-treatment with either 0.1% DMSO (labeled control) or 0.5 μM doxorubicin (labeled Doxo) for 1.5 hrs. Neutral comet assay was then performed as described in Materials and Methods. The average comet tail moments were plotted as histograms (mean±SEM). *p-value<0.001, t-test.

Doxorubicin-induced DNA damage could be due to either Top2-DNA covalent (cleavable/cleavage) complexes or ROS. As shown in FIG. 7A, doxorubicin-induced γ-H2AX was unaffected by the known ROS scavengers, vitamin C (100 μg/ml) and N-Acetyl Cysteine (NAC) (100 μg/ml). By contrast, as shown in FIG. 6B, the proteasome inhibitors, bortezomib (1 μM) and MG132 (4 μM), significantly reduced (more than 50% reduction, see lower panel for quantification) the γ-H2AX signal induced by doxorubicin and VP-16. Recent studies have suggested that proteasomal processing of VP-16-induced Top2-DNA covalent complexes results in the exposure of Top2-concealed DSBs. Thus, the involvement of proteasome in doxorubicin-induced γ-H2AX could implicate the involvement of Top2 in doxorubicin-induced DNA damage.

To test whether DSBs were indeed induced by doxorubicin and prevented by proteasome inhibitors, a neutral comet assay was performed. As shown in FIG. 7C, doxorubicin-induced comet tail moment, which reflects the amount of chromosomal DNA DSBs, was significantly reduced by co-treatment with either bortezomib (p-value<0.001, t-test) or MG132 (p-value<0.001, t-test). These results suggest that, similar to VP-16-induced DSBs, doxorubicin-induced DSBs are also Top2-mediated and proteasome-dependent.

### Example 8

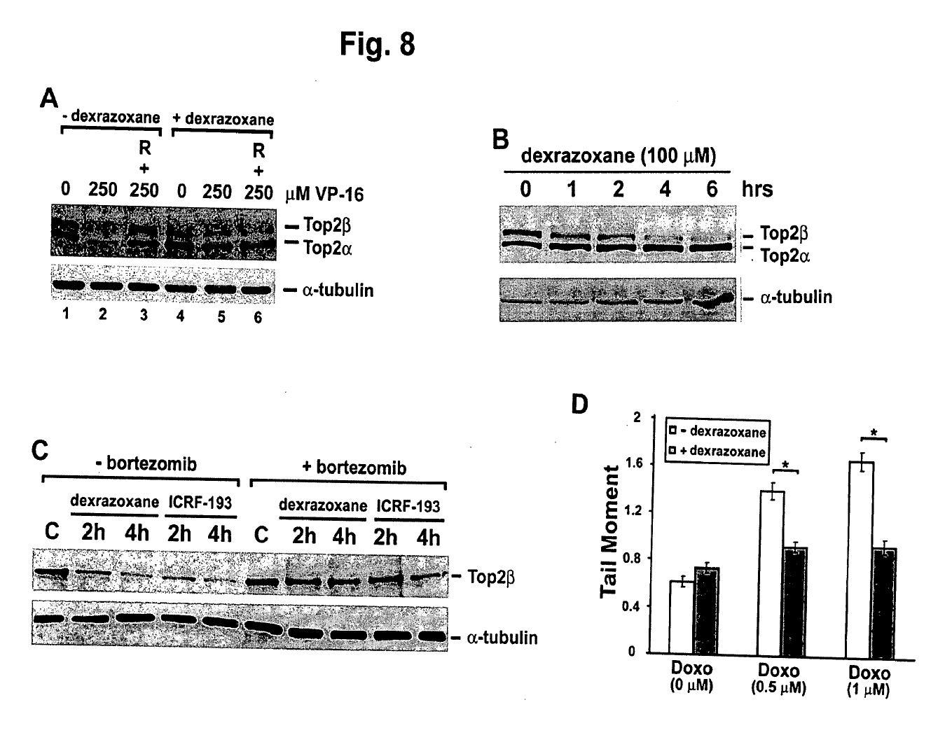

FIG. 8 Dexrazoxane induces proteasomal degradation of Top2β in H9C2 cardiomyocytes. A, Dexrazoxane antagonizes the formation of Top2α and Top2β-DNA covalent (cleavage) complexes. H9C2 cells were treated with VP-16 in the presence or absence of dexrazoxane (150 μM) for 15 min. The amount of Top2 cleavage complexes was measured by a band depletion assay as described in Materials and Methods. Cells were lysed either immediately or after reversal of the Top2 cleavage complexes (labeled R+250). Cell lysates were analyzed by Western blotting using anti-Top2α/Top2β or anti-α-tubulin antibody. B, 1.2×10⁵ H9C2 cells were treated with 100 μM dexrazoxane for indicated times (0, 1, 2, 4 and 6 hrs). Cells were then lysed and protein levels of Top2α, and Top2β isozymes were determined by Western blotting. C, H9C2 cells were treated with 0.1% DMSO (labeled C, for solvent control), dexrazoxane (100 μM) or ICRF-193 (50 μM) for 2 h or 4 h, in the presence or absence of the proteasome inhibitor, bortezomib (1 μM). Cell lysates were immunoblotted using anti-Top2β antibody. D, H9C2 cells were treated with ICRF187 (100 μM) for 4 hrs, followed by treatment with doxorubicin (Doxo, 0, 0.5 and 1 μM) for 1.5 hrs. Neutral comet assay was then performed as described in Materials and Methods. The average comet tail moments were plotted as histograms (mean±SEM). *p-value<0.001, t-test.

Recent studies have also shown that ICRF-193, can efficiently induce proteasome-mediated degradation of Top2β. Degradation of Top2β is also expected to reduce doxorubicin-induced DNA damage and doxorubicin cytotoxicity in H9C2 cells. Applicants tested the effect of dexrazoxane on the protein level of Top2β in H9C2 cardiomyocytes. As demonstrated in FIG. 8B, treatment of H9C2 cells with 100 μM dexrazoxane induced a time-dependent disappearance of the Top2β isozyme, while no significant effect on the level of the Top2α isozyme was observed. Similar to ICRF-193-induced degradation of Top2β, dexrazoxane-induced degradation of Top2β is proteasome-mediated. As shown in FIG. 8C, co-treatment of H9C2 cardiomyocytes with the proteasome inhibitor, bortezomib, abolished dexrazoxane-induced degradation of Top2β. These results suggest that dexrazoxane induces efficient proteasomal degradation of Top2β in H9C2 cardiomyocytes.

To test whether dexrazoxane-induced Top2β degradation could contribute to the protective effect of dexrazoxane on doxorubicin-induced DNA damage, H9C2 cardiomyocytes were pre-treated with dexrazoxane for 4 hrs to induce Top2β degradation and doxorubicin-induced chromosomal DNA DSBs were then measured by the neutral comet assay in the absence of dexrazoxane. As shown in FIG. 8D, dexrazoxane pre-treatment effectively reduced doxorubicin-induced comet tail moment (p-values<0.001, t-test). Together, these results suggest that dexrazoxane could protect doxorubicin-induced DNA damage at least in part through proteasomal degradation of Top2β.

### Example 9

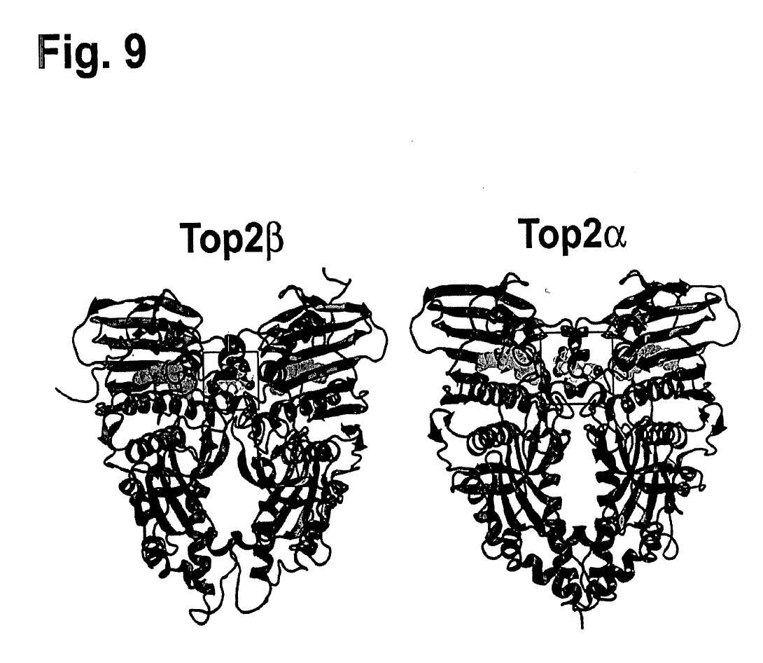

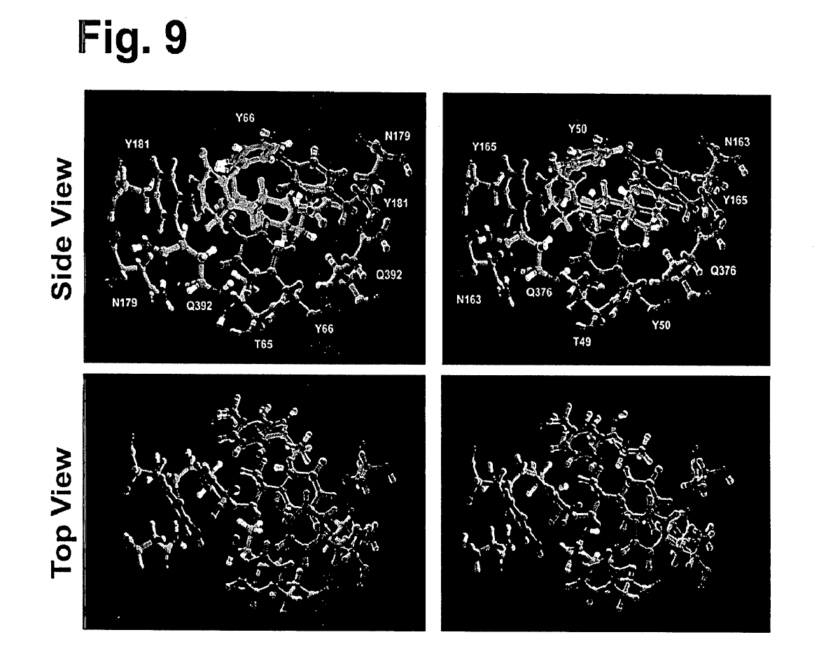

FIG. 9 Homology modeling of the N-terminal ATPase domain of human Top2α and Top2β in complex with dexrazoxane. Homology modeled structures of the ATPase domain of human Top2β and Top2α in complex with dexrazoxane are shown in the left and right panel, respectively. The Top2 isozyme dimers are symmetric with the separate protein chains indicated in red and blue (top panels). ADPNP (in green) and dexrazoxane (in CPK coloring) are shown using space-filling models. The dexrazoxane binding region (boxed in both top panels) is composed of residues from both chains at the dimer interface. The lower panels (both side view and top view) show the proximal residues in the dexrazoxane binding sites of human Top2β (left panels) and Top2α (right panels) in complex with dexrazoxane.

Dexrazoxane was shown to form a tight complex with the ATPase domain of human Top2β at the dimer interface. In addition, dexrazoxane forms various interactions with the same conserved amino acid side chains (see amino acids at the binding sites in FIG. 9, middle panel) at the binding site of human Top2β ATPase domain as those of the yeast Top2 ATPase domain. We have also performed homology modeling of the human Top2α (ATPase domain)-dexrazoxane complex. The overall structure of the complex is very similar to that of the human Top2β-dexrazoxane complex. Most strikingly, the various interactions between dexrazoxane and the amino acid side chains at the binding sites of the two human isozymes are identical (FIG. 9, middle and bottom panels). These modeling studies suggest that dexrazoxane can form a tight complex with both human Top2 isozymes.

### Example 10

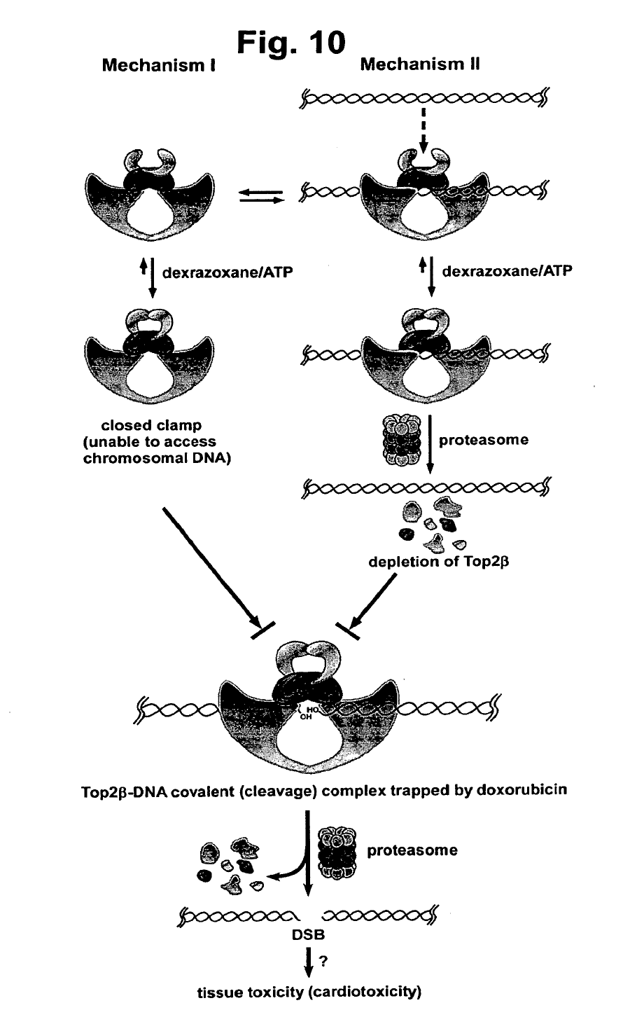

FIG. 10 Two proposed mechanisms for the antagonistic effect of dexrazoxane on doxorubicin-induced DNA damage. In this model, only the role of the Top2β isozyme is considered, which would mimic the situation in adult heart where Top2β, but not Top2α, is expressed. Top2β is shown to exist in two states, free Top2β (Mechanism I) and DNA bound Top2β (Mechanism II), at equilibrium. Dexrazoxane can bind to Top2β in either state. For Mechanism I, binding of dexrazoxane to free Top2β stabilizes the closed-clamp conformation of ATP-bound Top2β and thus prevents binding of Top2β (closed-clamp) to chromosomal DNA. Consequently, doxorubicin is unable to trap Top2β into cleavage complexes. For mechanism II, dexrazoxane binds to DNA-bound Top2β and stabilizes the closed-clamp conformation of ATP-bound Top2β, which triggers proteasomal degradation of Top2β (Top2β down-regulation). Top2β down-regulation results in depletion of Top2β and thus fewer doxorubicin-trapped Top2β cleavage complexes. The formation of doxorubicin-trapped Top2β cleavage complexes leads to DNA double-strand breaks (DSB) through proteasome-mediated processing, which, if not repaired, could contribute to cell death and possible tissue toxicity (e.g. cardiotoxicity).

The studies show that doxorubicin induces γ-H2AX, a key DNA damage signal reflecting primarily DNA DSBs, in H9C2 cardiomyocytes. Using this system, Applicants have demonstrated that the doxorubicin-induced DNA damage signal is unlikely to be the result of ROS-mediated DNA damage since vitamin C and NAC cannot attenuate this signal. Instead, several pieces of evidence suggest that the doxorubicin-induced DNA damage signal is primarily due to the formation of Top2β-DNA covalent complexes. First, doxorubicin-induced γ-H2AX was shown to be specifically abolished by proteasome inhibitors, MG132 and bortezomib. This result is suggestive of an involvement of Top2 since Top2-DNA covalent (cleavage) complexes, unlike other DNA damages (e.g. H₂O₂-mediated DNA damage), are known to require proteasome for their processing into DNA damage (DSBs). Indeed, doxorubicin is shown to induce chromosomal DNA DSBs in a proteasome-dependent manner (FIG. 7C and see the lower half of the diagram in FIG. 10 for the model). Second, doxorubicin-induced γ-H2AX is much attenuated in top2β^{-/-}MEFs compared to that in Top2β^{+/+} MEFs, suggesting the involvement of Top2β. Together, these results suggest the involvement of both Top2-DNA covalent complexes and proteasome in doxorubicin-induced DNA damage, which is consistent with the model that proteasome-mediated degradation of Top2-DNA covalent complexes exposes Top2-concealed DSBs.

The studies also show that dexrazoxane specifically abolished doxorubicin- and VP-16-induced, but not CPT- and H₂O₂-induced, γ-H2AX in H9C2 cardiomyocytes. Since both doxorubicin and VP-16, but not CPT and H₂O₂, are Top2 poisons, this result supports the conclusion that dexrazoxane antagonizes doxorubicin-induced DNA damage through its specific interference with Top2. Additional support for this conclusion comes from the use of ICRF-193 (structurally related to dexrazoxane, ICRF-187) which is a well characterized Top2 catalytic activity inhibitor. ICRF-193, which is known to be more potent than dexrazoxane in inhibiting Top2, is shown to be highly effective in antagonizing doxorubicin-induced γ-H2AX in H9C2 cardiomyocytes. The fact that both dexrazoxane and ICRF-193 antagonize the doxorubicin-induced DNA damage signal suggests not only the involvement of Top2 but a potential mechanism for their antagonism. Bis(2,6-dioxopiperazines) such as ICRF-193 and ICRF-159 are known to stabilize the closed-clamp conformation of ATP-bound Top2. It has been well documented that the closed-clamp conformation of Top2 interferes with the formation of Top2 cleavage complexes induced by Top2-directed drugs, possibly due to the inability of the closed-clamp form of Top2 to access chromosomal DNA. Consequently, dexrazoxane may antagonize doxorubicin-induced DNA damage through preventing the formation of Top2 cleavage complexes on chromosomal DNA (due to dexrazoxane-stabilization of the closed-clamp conformation of Top2 which is unable to access chromosomal DNA).

The identification of Top2β as the major target for doxorubicin-induced DNA damage has suggested a possible new mechanism for the antagonistic effect of dexrazoxane on doxorubicin-induced DNA damage. ICRF-193 is known to induce preferential degradation of the Top2β isozyme through a proteasome pathway, referred to as Top2β down-regulation. The reduced Top2β level in ICRF-193-treated cells is expected to decrease the amount of doxorubicin-induced Top2β cleavage complexes and hence reduce DNA damage. Indeed, the studies show that dexrazoxane, like ICRF-193, is highly effective in reducing the level of Top2β (but not Top2α) in H9C2 cardiomyocytes through the activation of a proteasome pathway (FIG. 8). Consequently, dexrazoxane is likely to antagonize doxorubicin-induced DNA damage through two mechanisms; 1) direct interference with the formation of Top2 cleavage complexes and 2) Top2β down-regulation.

The antagonistic effect of dexrazoxane on doxorubicin-induced DNA damage in H9C2 cardiomyocytes observed in the current study is relevant to the protective effect of dexrazoxane against doxorubicin cardiotoxicity in patients. It has been shown that the heart might be one of the tissues that prominently express the Top2β mRNA in adult mice. Interestingly, the Top2α mRNA is completely absent in the heart but still detectable in some other adult tissues such as the spleen and intestine. These findings indicate that Top2β is the only Top2 isozyme that is present in the adult heart and suggest that Top2β-targeting by doxorubicin could contribute to its toxic side effects (i.e. cardiotoxicity). In addition, it is known that Top2β can be detected in mitochondria and doxorubicin can accumulate in mitochondria that are abundant in the heart. These results suggest that Top2β-targeting by doxorubicin in both nuclei and mitochondria of cardiomyocytes could contribute to doxorubicin cardiotoxicity.

The current studies, therefore, may have relevance to doxorubicin cardiotoxicity. The two proposed mechanisms (see FIG. 10) for the antagonistic effect of dexrazoxane on doxorubicin-induced DNA damage may have interesting clinical implications. In mechanism I, dexrazoxane stabilizes the closed-clamp form of Top2 and thus prevents access of Top2 to chromosomal DNA. Consequently, doxorubicin is unable to trap Top2 on chromosomal DNA to form Top2-DNA covalent (cleavage) complexes. This mechanism is not Top2 isozyme-specific since dexrazoxane can stabilize the closed-clamp forms of both Top2α and Top2β. In fact, our homology modeling studies of the human Top2α and Top2β in complex with dexrazoxane have indicated that the dexrazoxane binding sites are the same for the two isozymes, with identical interactions between dexrazoxane and the various amino acid side chains. There are increasing evidence that the antitumor activity of Top2-targeting drugs is primarily due to Top2α-targeting in part due to the over-expression of Top2α in tumor cells. Consequently, dexrazoxane is expected to reduce the antitumor activity of doxorubicin through mechanism I.

By contrast, dexrazoxane can down-regulate the Top2β isozyme specifically through mechanism II (FIG. 10). Through this mechanism, dexrazoxane is expected not to have a major impact on the Top2α isozyme level and hence the antitumor activity of doxorubicin (and other Top2-targeting drugs). If indeed, dexrazoxane, used under the current clinical protocol, prevents doxorubicin cardiotoxicity through both mechanisms, strategies should be developed to prevent mechanism I and favor mechanism II. For example, proper timing of dexrazoxane pretreatment during doxorubicin-based chemotherapy may change the contribution through these two mechanisms.

That Top2β-targeting is primarily responsible for doxorubicin cardiotoxicity has significant clinical implications. This provides the necessary rationale for developing Top2α-specific anticancer drugs to prevent tissue toxicities (i.e. cardiotoxicity) in patients receiving Top2-based chemotherapy. It is also noteworthy that the involvement of proteasome in Top2β-mediated DNA damage is a novel approach for preventing doxorubicin cardiotoxicity through the combined use of bortezomib (or other proteasome inhibitor) and doxorubicin.

We claim:

1. A method for treating a patient with neoplasia comprising administering a therapeutically effective amount of a compound to the patient, wherein the compound preferentially poisons the Top2α isozyme compared to the Top2β isozyme.

2. The method of claim 1 wherein the neoplasia is selected from a group of leukemias, colon cancer, pancreatic cancer, lung cancer, prostate cancer, Wilms' tumor, neuroblastoma, soft tissue sarcoma, bone sarcoma, lymphoma, bladder cancer, breast cancer, stomach cancer, lung cancer, ovarian cancer, thyroid cancer, gastric cancer, colorectal cancer, pancreatic cancer, brain cancer, testicular cancer, glioblastoma multiforme, Hodgkin's disease, Ewing's sarcoma, bronchogenic carcinoma and multiple myeloma.

3. A method for treating a patient with neoplasia comprising:
a) administering a therapeutically effective amount of a Top2 inhibitor to a patient with neoplastia, wherein the inhibitor preferentially inhibits Top2β isozyme over Top2α isozyme;
b) administering a therapeutically effective amount of at least one Top2 poison to the patient;
wherein the Top2 inhibitor is administered at least 2 hours prior to administration of the Top2 poison.

4. The method of claim 3 wherein the topoisomerase II activity inhibitor is selected from a group of ICRF-187, ICRF-193, ICRF-159, ICRF-154, and prodrugs and metabolites thereof.

5. A method for treating a patient with neoplasia comprising: administering a therapeutically effective amount of a Top2 poison and a therapeutically effective amount of a proteasome inhibitor to said patient.

6. The method of claim 5 wherein the proteasome inhibitor comprises bortezomib.

7. A method of identifying an anti-neoplastic compound comprising:
a. evaluating the compound for its ability to poison Top2α isozyme;
b. evaluating the compound for its ability to poison Top2β isozyme; and
c. selecting a compound that preferentially poisons the Top2α isozyme over the Top2β isozyme
